(19) United States
(12) Patent Application Publication
(10) Pub. No.: US 2010/0129453 A1
(43) Pub. Date: May 27, 2010

(54) EMULSIONS COMPRISING RUBBER ARABICUM

(76) Inventor: Daniel Strasser, Gossau (CH)

Correspondence Address: LADAS & PARRY LLP, 26 WEST 61ST STREET, NEW YORK, NY, 10023, US

(21) Appl. No.: 12/311,075

(22) PCT Filed: Sep. 18, 2007
(86) PCT No.: PCT/CH2007/000455

§ 371 Date: Dec. 8, 2009

(30) Foreign Application Priority Data
Sep. 22, 2006 (CH) 1510/06

(51) Int. Cl. A61K 9/14; A61K 47/00; A61K 38/28; A61K 8/02; A01N 25/12; A61P 3/10; A61Q 90/00; A01P 15/00; A23L 1/30

(52) U.S. Cl. 424/489; 514/782; 514/3; 426/61

(57) ABSTRACT

The invention relates to micelles, micellar solutions and pre-concentrates of emulsions comprising active ingredient and Gum Arabic; products comprising such micelles or pre-concentrates of emulsions, respectively; processes for manufacturing micelles, pre-concentrates of emulsions and products.

The invention relates to micelles, micellar solutions and pre-concentrates of emulsions comprising active ingredient(s) and und Gum Arabic; products comprising such micelles, micellar solutions and pre-concentrates of emulsions respectively; processes for manufacturing micelles, micellar solutions, pre-concentrates of emulsions and products.

A typical problem when applying active ingredients is its insufficient bioavailability. Bioavailability denotes that part of an active ingredient which is available to the systemic circuit of a human/animal/plant unmodified. It denotes how fast and to which extend the active ingredient is resorbed and made available at the site of pharmaceutical location. For active ingredients, which are administered intravenous (“i.V.”), the bioavailability is 100%, by definition. The absolute bioavailability denotes the bioavailability of a substance applied in any manner (e.g. peroral) in comparison to the intravenous application. The bioavailability observed after oral application is also termed oral bioavailability. It is of particular importance to improve oral bioavailability, as the oral application of active ingredients is preferred over other dosage forms.

It is known that by addition of excipients the bioavailability of active ingredients may be improved. Such compositions, comprising active ingredient(s) und excipients are often termed “formulations” of “formulations of active ingredients”. In principle, numerous known excipients, such as (poly)sorbates (Tween®), would be suitable to provide formulations with improved bioavailability. However, it has been shown that such excipients are little suitable, due to other disadvantages, such as physiological concerns and/or disadvantageous taste.

It is further known that Gum Arabic (E414; CAS. Nr. 9000-01-5, a known natural product/natural product mixture is used in foodstuffs and for the formulation of pharmaceuticals. The known formulations may effect, depending on its kind, a solubilization, micellization and/or disperzation of active ingredients. In many cases the result is not, or only partly, satisfying. GB824912 discloses, for example, a process for stabilizing Vitamin E by use of Gum Arabic, wherein an emulsion with particle size below 2 um is produced ad interim. However, such emulsion is not stable and is thus used for the manufacture of a solid end-product only.

It is further known that emulsions—contrary to micellar solutions—are only stable up to a characteristic concentration of the micelles. Below a certain concentration (critical micelle concentration, cmc) the emulsion falls apart the phases separate. By this effect, the bioavailability of the emulsified active ingredient is reduced to virtually 0%.

The application of many active ingredients, whose potency is known, causes problems, as its solubilization and stabilization is difficult. Co-enzyme Q10 is referred to as an example of such an active ingredient. The opportunity of solubilizing and simultaneously stabilizing such active ingredient, or an active ingredient of similar quality, in micelles with the same or even improved loading would offer undreamed-of possibilities.

Active ingredients or medicaments treated in such a way would not only be suitable as capsules for oral administration, one could also blend these active ingredients to a drink, such as a sports drink. To make available targeted active ingredients, which are until now liposoluble, in drinks would offer, due to the fact that they are soluble and stable therein, significant advantages for the consumer. A further important active ingredient, whose application was found difficult, is insulin. To achieve an appropriate bioavailability, it needs to be administered i.V., which is disadvantageous for the patient. For this reason, an oral dosage form would be desirable. Next to medical active ingredients there is a number of further active ingredients which could not, or not sufficiently, solubilized, dispersed or stabilized.

Besides insufficient bioavailability of the formulation, or the products containing such formulations, are to be mentioned insufficient shelf life, disadvantageous optical properties (like clouding and/or staining) or disadvantageous physiological properties (like side effects of the excipients, smell, taste).

There is a need to provide formulations for active ingredients and the manufacturing thereof, which overcome one or more of the disadvantages identified herein. In particular, there is a need in excipients, which effect, allow, support or facilitate the solubilization, micellizaton and disperzation. Ideally, such excipients are approved (i.e. already contained in lists of pharmaceuticals, or foodstuffs or similar registers), as classified harmless and no objections or reservations are expected from health authorities, consumers organizations or other interested parties. But not only the pure solubilization and disperzation is of importance, but also the stabilization of an active ingredient (which is optionally solubilized and/or micellized) to develop/improve its effect, or resorption respectively, as long and complete as possible.

Now, the present invention relates to an improved process to solubilize, to dispergize, to micellize and optionally stabilize active ingredients and thereby to provide products, which resolve one or more of the above identified problems. Such active ingredients may be, for example lipophilic active ingredients in hydrophilic milieu as well as hydrophilic active ingredients in lipophilic and hydrophilic milieu, in particular in aqueous milieu. By solubilizing, dispersing and micellizing of active ingredients as described herein, a stabilizing thereof is achieved in hydrophilic milieu. Finally, the invention comprises the use of such compounds and active ingredients. The present invention further relates to active ingredients contained in micelles, pre-concentrates of emulsions comprising such micelles and products containing micelles or pre-concentrates of emulsions.

The above identified objectives are achieved according to the independent claims. The dependent claims provide advantageous embodiments thereof. The general, preferred and particularly preferred embodiments and ranges provided in the context of the present invention may be combined at will. Also, selected definitions, embodiments may not be applicable or irrelevant. In the following, various aspects of the invention are explained in detail.

In a first aspect, the invention relates to micelles having a diameter of 2-300 nm, comprising i) one or more active ingredients; ii) Gum Arabic; iii) optionally one or more excipients. It was surprisingly found that active ingredients, embedded in such micelles show a significantly improved bioavailability.

In the context of the present invention, by “active ingredient”, a natural, identical to a natural or synthetic compound is understood, which causes a physiological effect within or at a human, animal or plant. Active ingredients may be distinguished according to its function in the groups of food additives, pharmaceutically active ingredients (for human or animal), cosmetically active ingredients, agricultural active ingredients, whereby selected active ingredients may be assigned to more than on group, as the case may be. In the context of the present invention, such active ingredients are preferably used, which are difficult to formulate in other ways and/or which possess low bioavailability. Such active ingredients typically have a water-solubility of less than 10 g/l (20° C.; neutral medium) or of less than 100 g/l (20° C., physiological medium (e.g. synthetic body fluid)). The molecular mass of such active ingredients may vary over a broad range and comprises “small molecules” starting with a molecular mass of about 50 g/mol up to macro molecules of about 50.000 g/mol. The term active ingredient shall, in addition to a single active ingredient, also comprise the combination of more than one active ingredient, whereby each single active ingredient of such combination possesses the properties described herein.

“Food additives” comprise compounds which are approved for human consumption and may be added to food products. Food additives are known to the skilled person and may be, for example, identified in des regulation of the EDI regarding additives approved for food products (“Zusatzstoffverordnung”, ZuV) dated 23 Nov. 2005. In the context of the present invention, emphasis is given particularly to the class of vitamins (e.g. vitamin A, E), of fatty acids (e.g. unsaturated fatty acids, such as Omega-3-fetty acids, Omega-6-fetty acids), of enzymes (e.g. from the group of flavonoides) and of co-enzymes e.g. co-enzyme Q10) and coloring agents (e.g. carotenoides such as beta-carotene, lycopene and luteine).

“Pharmaceutically active ingredients” comprise compounds which are approved for the prevention and/or therapy of diseases of humans or animals. Pharmaceutically active ingredients are known to the skilled person and may be identified e.g. in the “Orange Book” or the “Roten Liste”. In the context of the present invention, emphasis is given to proteinogenic active ingredients (e.g. natural or genetically engineered insulin).

“Cosmetically active ingredients” comprise compounds which are applied in the field of cosmetics and are responsible for a physiological effect (e.g. to the skin/hair/nails) or an optical effect (e. g. a coloring). Cosmetically active ingredients are known to the skilled person and may be identified e.g. according to INCI. In the context of the present invention, emphasis is given to the class of cosmeticals.

“Agricultural active ingredients “comprise compounds which are applied in the field of agriculture, forestry and gardening. Agricultural active ingredients are known to the skilled person an may be identified e.g. in the “Pesticide Manual”. In the context of the present invention, emphasis is given to the group of herbicides, fungicides, insecticides and growth regulators.

It shall be noted that active ingredients may be grouped in more than one of the above identified categories, for example food additive and pharmaceutical (e.g. because a pharmaceutical effect is yet not discovered or under dispute). In the context of the present invention, it is sufficient if the skilled person is in the position to assign an active ingredient to at least one of the above defined classes.

By “Gum Arabic” the known, commercially available natural product is understood, which is also registered as E414 and CAS 9000-01-05 respectively. Due to its natural provenience, its composition may vary, whereby the various mixtures and grades of purity are comprised (“natural Gum Arabic“). Further, the term comprises poly-arabinic acid and its derivatives, particularly acid earth alkali- and alkali salts, which are available by synthetic or semi-synthetic approaches (“synthetic Gum Arabic”).

By “excipients”, such compounds are understood, which show a positive effect to the micelle, the pre-concentrate of emulsion and/or the ready-to-market product. A group of excipients are solubilizers, which reduce the melting point of Gum Arabic or which form a homogeneous phase with Gum Arabic below its melting point, respectively. Typical examples are compounds selected from the group of polyoles, such as glycerin, propylene-glycol, polyethylene-glycol 400 etc. Such excipients are preferably added, if the active ingredient to be manufactured is thermally labile at the melting point of Gum Arabic (e.g. proteins).

The term “micelle” is known to the skilled person. It denotes a structure possessing a core and a shell in a certain size and a shape that is described as irregular ellipsoid or spherical. Typically, micelles possess a diameter of below 500 nm and possess, due to its manufacture, a certain size distribution. Size and Size distribution may be determined by optical methods (e.g. microscopy). In the context of the present invention, micelles preferably show a diameter of 2-300 nm; particularly preferably 10-100 nm. Typically, at least 66%, preferably 75% most preferably 80% of the micelles are within the above identified interval. The core of the micelle essentially consists of the active ingredient(s), die shell essentially consists of Gum Arabic. As a micelle is a dynamic structure, the borders (core/shell and shell/surrounding are more or less distinguished or blurred. Further excipients, if applicable, are located either predominately in the core or predominately in the shell of the micelle, depending on its distribution coefficient.

In a preferred embodiment, the present invention relates to micelles as described above, comprising one or more Active ingredients having a solubility of less than 1 g/l, selected from the group of food additives, pharmaceutically active ingredients, cosmetically active ingredients and agricultural active ingredients.

In a further preferred embodiment, the present invention relates to micelles as described above, wherein the active ingredient is coenzyme Q 10 and/or vitamin E.

In a further preferred embodiment, the present invention relates to micelles as described above, wherein the active ingredient is insulin.

In a further preferred embodiment, the present invention relates to micelles as described above, wherein the active ingredient is idebenone.

In a further preferred embodiment, the present invention relates to micelles as described above, wherein the active ingredient is tocotrienole. In the context of the present invention, by “tocotrienole” is meant a natural existing mixture of tocotrienole, as well as a mixture enriched in one of the forms alpha, beta, delta, gamma, as well as pure alpha-, beta- delta- or gamma-tocotrienole.

In a further preferred embodiment, the present invention relates to micelles as described above, wherein the active ingredient comprises one or more omega-3-Fetty acids.

In a further preferred embodiment, the present invention relates to micelles as described above, comprising one or more excipients selected from the group of polyoles.

In a further preferred embodiment, the present invention relates to micelles as described above, wherein the ratio of active ingredient(s):Gum Arabic is in the range from 20:1 to 1:10 mass-%, preferably 10:1 to 1:1. In general, it is desirable to reduce the amount of Gum Arabic and to maximize the amount of active ingredient. On the other hand, having a view to the aimed use, a sufficient stability and bioavailability must be ensured. The optimal ratio of active ingredient(s):Gum Arabic may be determined by simple experiments. The properties of the active ingredient, particularly is solubility, have an influence on the above identified ratio. In comparison to known formulations of active ingredients, it is possible to positively influence the ratio active ingredient(s):Gum Arabic on the one hand and to obtain a narrow size distribution of micelles on the other hand, when applying the process as described below. The micelles according to the invention are thus stable, enable a high bioavailability and an advantageous release-profile of the active ingredient.

In a second aspect, the invention relates to pre-concentrates of emulsions and micellar solutions respectively, comprising i) a lipophilic phase, which contains micelles as described above, and ii) an aqueous phase. Typically, micelles are not stable per se, but dispersed in a further phase. Often, such pre-concentrates of emulsions/micellar solutions are obtained directly during manufacturing. Such pre-concentrates of emulsions/micellar solutions may be either delivered as trade products to the downstream industry (for manufacturing “products”, see below) or directly further processed. Accordingly, the further processing of pre-concentrates of emulsions/micellar solutions for the manufacture of products as defined below, is also comprised.

Pre-concentrates of emulsions may be characterized by the type of emulsion, micellar concentration, -size, -size distribution. Type: Pre-concentrates of emulsions may be characterized as oil-in-water (O/W; die homo-geneous phase is aqueous) or water-in-oil (W/O; die disperses phase is aqueous). Preferred are O/W-emulsions. Concentration: Typically, in pre-concentrates of emulsions, the micellar concentration is higher than in products. With respect to the present invention, an ratio of lipophilic phase:aqueous phase proved to be advantageous in the range of 1:10 to 1:0.5. Size and size distribution of the micelles are already explained in the context of the description of micelles, whereupon it is referred back to.

Thus, in an advantageous embodiment, the present invention relates pre-concentrates of emulsions and micellar solutions respectively as described above, wherein the ratio of lipophilic phase : aqueous phase is in the range of 1:10 to 1:1.

The “lipophilic phase” of the pre-concentrates of emulsions and micellar solutions respectively essentially consists of, preferably consists of, micelles as described herein.

In one embodiment, the “aqueous phase” consists (only) of water. For thermodynamic reasons, the aqueous phase will always contain Gum Arabic and active ingredient and optionally further excipients present in the micelle to a certain extend; for simplicity, it is however referred to as a “pure” aqueous phase. The water used may have various grades of purity (e.g. purified, de-ionized; for i.V. applications, . . . ), depending on the intended further use. Such grades of purity are comprised by the invention.

In a further embodiment, the aqueous phase contains further components. Such components may be used to influence the pH-value (acids, bases, buffer), to influence the ionic strength (buffer, salts) or to influence rheological properties (thickener). One or more of such components may be present. Such components are known to the skilled person and may be identified e.g. in Fiedler, Lexikon der Hilfsstoffe für Pharmazie, Kosmetik and angrenzende Gebiete (1989).

In a further embodiment, the invention relates to pre-concentrates of emulsions, comprising a transparent gel based on at least one Gum Arabic and an active ingredient solubilized and micellelized therein, whose consistency is between semi-solid (“Aspic-type”) and liquid.

In a third aspect, the invention relates to products from the group of foodstuffs, cosmetics, pharmaceuticals and plant protection products which comprises micelles as described above, or one or more pre-concentrates of emulsions as described above, or one or more micellar solutions as described above.

Such products may possess a wide range of appearances. For illustrative purposes, the following products are identified. i) Liquids: as drink in the field of food; as solution, drops, syrup in the field of pharmaceuticals; as spray or solution in the field of cosmetics, as sprayable solution in the field of agriculture. ii) Gel or jelly: as sandwich spread, for application to the body (pharmaceutical, cosmetics). iii) Crèmes or pasts: In bakery products, sandwich spread, in snacks; as crème (pharmaceutical, cosmetics). As it becomes apparent from the above-given enumeration, the products may a product ready for marketing (e.g. for liquids) or be part of a marketed product (e.g. crème in a bakery product)

The pre-concentrates of emulsions/micellar solutions may be added to the products as described above in various amounts. The amount depends on, inter alia, the desired result/the required amount of supplied active ingredient. Such amount may be determined by simple routine experiments. As the pre-concentrates of emulsions/micellar solutions ensure an improved bioavailability, it is in general possible to use comparatively lower amounts; which is considered a significant advantage. It was found that, due to its properties, the Pre-concentrates of emulsions/micellar solutions may be added to virtually all products, without negatively influence the products. Liquids substantially remain clear and stable over a long period of time. Further, the products are not negatively influenced in its sensory properties (particularly taste). Further, no disadvantageous physiological properties are known. Such positive properties are considered a significant advantage from the perspective of marketing as well as from the perspective of the end-user.

In a preferred embodiment, the present invention relates to a product from the group of foodstuffs, particularly a drink, which contains coenzyme Q10 and/or vitamin E.

In a preferred embodiment, the present invention relates to a product from the group of foodstuffs, particularly a drink, or of pharmaceuticals, particularly a liquid, oral administrable formulation, which contains idebenone.

In a preferred embodiment, the present invention relates to a product from the group of foodstuffs, particularly a drink, or of pharmaceuticals, particularly a liquid, oral administrable formulation, which contains tocotrienole.

In a preferred embodiment, the present invention relates to a product from the group of pharmaceuticals, particularly a liquid, oral administrable formulation, which contains insulin.

In a further embodiment, the present invention relates to a product, comprising Gum Arabic and a micellelized active ingredient which is solubilized, dispersed and/or stabilized therein, wherein the consistency of the product is between semi-solid and liquid.

In a further embodiment, the present invention relates to a product comprising a transparent gel which contains i) Gum Arabic and/or Gum Arabic/Glycerin-Ester and/or one of its substitutes and/or derivatives, and ii) one or more solvents from the group of water, glycerin, propylene-glycol, polyethylene-glycol 400, ethanol, Macrogol 400, iso-propanol and further one or more lipophilic or hydrophilic active ingredients solubilized, dispersed and stabilized therein.

The consistency of products may vary in a broad range. Thus, solid (cuttable, brittle) semi-solid (e.g. Aspic-like) and liquid (thin fluid like water or syrup-like) products are comprised. Products are not necessarily homogeneous (crèmes, foams) or may be encapsulated (dragees).

In a fourth aspect, the present invention relates to a process for manufacturing pre-concentrates of emulsions/micellar solutions comprising the step of i) providing a homogeneous phase either by melting Gum Arabic at 40-60° C. or by dissolving Gum Arabic in one or more excipients at 0-60° C.; ii) metered addition of active ingredient, which is optionally dispersed in an aqueous phase, to the homogeneous phase; iii) optionally further addition of an aqueous phase to the obtained stirred reaction mixture, whereby at least in on the the reaction steps an aqueous solution is added and whereby the temperature of the active ingredient, which is optionally dispersed in water, is ±10° C. of the homogeneous phase.

It was found that pre-concentrates of emulsions, micellar solutions may be manufactured in a simple and safe manner, even for active ingredients otherwise difficult to formulate, according to the process described herein. It is particularly surprising that micellar solutions are obtained, when this process is used. Such micellar solutions are stable, even in high dilutions of the micelles in the continuous phase, and thus ensure bioavailability: Upon application, the micelles may be arbitrary diluted with water or aqueous liquids (e.g. gastric juice, chyle or sweat or intracellular liquid), without stripping the emulgators from the surface of the micelles thereby enabling direct contact of water and active ingredient, which would lead to a break of the emulsion. For this reason, the active ingredient may be transported unchanged to the site of action by the micelles through membranes in the oral or the dermal application (skin or mucosa). This positive effect is proven in an impressing manner by the following examples. The opportunity of providing active ingredients in low dosages is important, as many active ingredients shall be added to food products in low amounts for cost reasons and/or as legal provisions allows a certain (maximum) dosage only.

The invention thus relates to processes for solubilizing, dispersing, micellizing and stabilizing of active ingredients, to pre-concentrates of emulsions, micellar solutions and products solubilized, dispersed, micellized and stabilized according to this process as well as the use of such pre-concentrates of emulsions and products.

By the term “Solubilizing” is meant, in the context of the present invention, the transfer of a lipophilic substance into a hydrophilic substance.

By the term “Dispersing” is meant, in the context of the present invention, the blending of different compounds by means of shear force.

By the term “Micellizing” is meant, in the context of the present invention, the transfer of active ingredient in micelles.

By the term “Stabilizing” is meant, in the context of the present invention, a process which establishes a system wherein the active ingredient remains unchanged over a longer period of time (particularly 1 day to 1 year, preferably 5-200 days).

The above described process is thus suitable for solubilizing, dispersing and stabilizing an active ingredient in micelles, characterized in that Gum Arabic and active ingredient are thoroughly dispersed, whereby the mixture thereof is covered by water of similar temperature afterwards and the spontaneously formed gel formed thereby is homogenized.

The invention particularly relates to a process as described above characterized in that the aqueous phase consists of water.

The invention particularly relates to a process as described above characterized in that the excipients are selected from the group of polyoles.

In a further embodiment, the present invention relates to a process for solubilizing, dispersing, micellizing and stabilizing of active ingredients, wherein Gum Arabic is used and the active ingredient to be treated is thoroughly dispersed in the till then resting suspension, whereby said is covered by water of the same temperature and the thus spontaneous formed gel is homogenized.

In a further embodiment, the present invention relates to a process for solubilizing, dispersing, micellizing and stabilizing of active ingredients, characterized in that Gum Arabic is molten and the active ingredient to be treated is thoroughly dispersed in this suspension, whereby after addition of the compound to be treated into the dispergate, the same is covered with water and the whole unit is homogenized again.

In a further embodiment, the present invention relates to a process for solubilizing, dispersing, micellizing and stabilizing of active ingredients, characterized in that Gum Arabic is dissolved in an excipient at room temperature, the temperature of the dispergate is maintained, the active ingredient is dispersed therein, the obtained melt is covered with water of the same temperature, the melt is homogenized whereby a slightly turbid and transparent gel is obtained.

In a further embodiment, the present invention relates to a process for solubilizing, dispersing, micellizing and stabilizing of active ingredients, characterized in that Gum Arabic is dissolved at room temperature; the mixture of compounds is lowered by addition of one or more excipients selected from the group comprising water, glycerin, at the same temperature; than the (thermo-labile) active ingredient to be solubilized, particularly insulin, is dispersed in the melt obtained; the melt is covered by water of the same temperature; the melt is homogenized; whereby a transparent gel is obtained.

As it becomes apparent from the explanations given above, an appropriate control of temperature favors the process. By the term “same temperature” and “constant temperature” is meant a temperature which is essentially the same. Such may vary depending on further process parameters, equipment design and substances used. Typically, a temperature range of ±10° C. is advantageous; particularly advantageous is a temperature range of ±5° C. Correspondingly, room temperature denotes 22° C.±10° C., preferably ±5° C.

As a further important process step it was found that the melt of Gum Arabic, containing therein the dissolved active ingredient, is immediately covered with a sufficient thick layer of water having about the same temperature. By this measure, a transparent gel is spontaneously formed below the water. Without such coverage with water of the same temperature, the melt hardens and is can not or can almost not be applied in this form. Therefore, the melt should, in its liquid state, be covered or lavished respectively, with water of about the same temperature. By using cold water, gel formation also takes place, but in this case a dispersion of the active ingredient will take place predominantly. After water of the same temperature was added and coverage of the melt is obtained—the water phase is located above the melt—gel formation commences and the gel grows into the water surface upwards, as it absorbs water. This gel formation, observable from the outside, is supported by the immediately contacting of water and melt, for example by controlled stirring. The gel adopts a micellar structure and shows the consistency of a thin solution. Gels having a droplet diameter of less than 40 nm are obtainable, on which light is not refracted and which are thus clear and transparent. This is particularly surprising, as for example 10% of a liposoluble active ingredient and about 10-20% Gum Arabic are contained. These smallest lipid droplets remain thermo-stable, such that on the one hand no coalescence of the lipid droplets occurs, even when boiling the gel and on the other hand the addition of water does not alter the micellar structure. The consistency is syrup l like or less. The gel is homogenized by stirring and diluted to a suitable viscosity by addition of water or water-solvent mixtures. If homogenization takes place under high shear force, this is disadvantageous with regards to gel formation. In such case, the obtained gel does not become transparent, which in turn means that along with solubilization also disperzation took place. When stirring takes place by using normal knifes—such as a “Stefan-Stirrer” (which consists of a rotary axis perpendicular to the body of the vessel and sharp knifes perpendicular to this axis) or a “Dissolver-Stirrer—which recurring cuts the material to be stirred, an optically pure, nice and transparent gel is readily obtained. In such gel, formation of seed crystals is significantly slowed down when compared to a liquid.

Below, the process for solubilizing and stabilizing of an active ingredient is still further explained. It was found there is a difference whether i) Gum Arabic is simply added to an aqueous phase followed by addition and mixing of an active ingredient (hoping that it is protected and stabilized by homogenizing the mixture) or ii) Gum Arabic and active ingredient are merged in a well-directed manner. Without being bound to theory, it is considered particularly important that Gum Arabic and the active ingredient to be treated are merged on a molecular basis. When simply mixing according to i), the effect of solubilizing and dispersing and stabilizing is obtained for a view per cent of the added active ingredient only. Provided Gum Arabic is not polluted with solvents it is liquid at room temperature. It was found that the majority of lipophilic and hydrophilic active ingredients form a homogenous phase with Gum Arabic. Without being bound to theory, such way of solubilization (wherein Gum Arabic and active ingredient to be solubilized are merged on a molecular or quasi-molecular basis) is considered an important element for improving the ratio of Gum Arabic to active ingredient. In return, much active ingredient may be enwrapped with less Gum Arabic. As a result, side effects of excipients are suppressed, as they are often added in large or very large excess in relation to the active ingredient in question.

In a fifth aspect, the invention relates to a process for manufacturing a product as described herein, characterized in that a pre-concentrate of emulsion/a micellar solution as described herein is blended with further components of the product. The addition of the pre-concentrate of emulsion may take place at various stages during the manufacture of the product. Placement and distribution of pre-concentrate of emulsion into the product may take place according to known methods and existing equipment. Such flexibility is considered an advantage. When producing drinks, this may be, for example, the last step in production. Generally, production-related consideration will prevail when choosing a suitable process of manufacturing the product. Although micelles and pre-concentrates of emulsion according to this invention are temperature stable, it was found advantageous, not to expose such pre-concentrate of emulsion towards high temperatures for a prolonged period of time.

The examples provided below shall further illustrate the invention, without limiting it.

1. Manufacture of a pre-concentrate of emulsion: 200 g Gum Arabic are placed first, combined with 200 g of PEG400 and homogenized at 40° C. A suspension containing 20 g of CoQ10 (ubiquinnone) in 200 g of water is heated to 40° C. and added during a period of 10 min while vigorously stirring. Afterwards, the obtained mixture is covered with 380 g of water having a temperature of 50° C. The reaction mixture obtained is, after cooling and degassing, a clear and transparent, golden yellow liquid.

2. Manufacture of a product: 1 g solution of the previous reaction are added to 500 g of a commercial drink (lemonade of brand Actilife Q10) and briefly stirred. No change in taste is observable. The product does not alter within 180 days. The aging was confirmed by HPLC via accelerated aging by light and/or temperature.

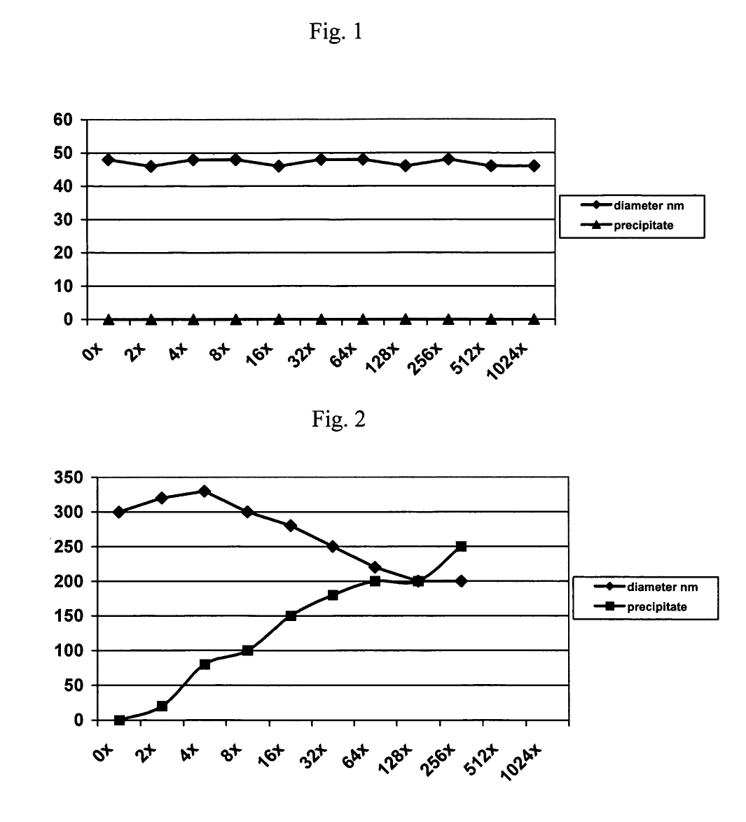

3. Stability tests: Coenzyme Q10 in micellar solution, manufactured according to the process described above, was exposed to a dilution series in a beaker under stirring, each using twice the amount of water. In this test, a sample of the following dilution was examined by microscopy, an average size of 50 nm was measured and gaps in micellar shells were investigated. The values provided in FIG. 1 relate in each case to the micellized active ingredient concentration in the surrounding fluid. FIG. 1 shows a micellar solution according to the invention having 2% Co-enzyme Q10 at neutral pH; In comparison, FIG. 2 shows an emulsion having the identical components but produced according to a standard process, also at neutral pH. For the micellar solution according to the invention the values remain unchanged upon lowering the pH value to 1.5%, while the standard emulsion breaks down to 100%, already at a pH of 6.8.

These results show that a micellar solutions produced according to the inventive process result in products which are much more stable towards dilution and pH lowering when compared to standard processes for emulsifying.

1. Composition in the form of micelles having a diameter of 2-300 nm, comprising
i) one or more active ingredients;
ii) Gum Arabic;
iii) optionally one or more excipients.

2. Composition according to claim 1, comprising one or more Active ingredients each having a water solubility of less than 1 g/l, selected from the group of food additives, pharmaceutically active ingredients, cosmetically active ingredients and agricultural active ingredients.

3. Composition according to claim 1, comprising one or more excipients selected from the group of polyoles.

4. Composition according to claim 1, wherein the active ingredient is co-enzyme Q10 and /or vitamin E.

5. Composition according to claim 1, wherein the active ingredient is insulin.

6. Composition according to claim 1, wherein the active ingredient is selected from the group consisting of
i) omega-3-fetty acids and/or omega-6-fetty acids; and/or
ii) flavonoids; and/or
iii) carotenoids.

7. Composition according to claim 1, wherein the ratio of active ingredient(s):Gum Arabic is in the range from 20:1 to 1:10 mass-%.

8. Pre-Concentrate of emulsion comprising
a) a lipophilic phase, which comprises a composition according to claim 1, and
b) an aqueous phase.

9. Pre-Concentrate of emulsion according to claim 8, wherein the ratio of lipophilic phase: aqueous phase is in the range of 1:10 to 1:0.5.

10. Micellar solution comprising
a) a lipophilic phase, which comprises a composition according to claims 1, and
b) an aqueous phase.

11. Micellar solution according to claim 10, wherein the ratio of lipophilic phase: aqueous phase is in the range of 1:10 to 1:0.5.

12. Process for manufacturing a Pre-Concentrate of emulsion or a micellar solution said preconcentrate or micellar solution comprising
a) a lipophilic phase, which comprises a composition according to claim 1 and
b) an aqueous phase
which process comprises the steps of:
(i) providing a homogeneous phase either by melting Gum Arabic at 40-60° C. or by dissolving Gum Arabic in one or more excipients at 0-60° C.
(ii) metered addition of active ingredient, which is optionally dispersed in an aqueous phase, to the stirred homogeneous phase
(iii) optionally further addition of an aqueous phase to the obtained stirred reaction mixture,
whereby at least in on the reaction steps an aqueous solution is added and
whereby the temperature of the active ingredient, which is optionally dispersed in water, is ±10° C. of the homogeneous phase.

13. Process according to claim 12, characterized in that the aqueous phase consists of water.

14. Process according to claim 12, characterized in that the excipients are selected from the group of polyoles.

15. Process for manufacturing a micallar solution comprising
a) a lipophilic phase, which comprises a composition according to claim 1 and
b) an aqueous phase
characterized in that the pre-concentrate of emulsion containing said components is blended with further components.

16. Product from the group of foodstuffs, cosmetics, pharmaceuticals and plant protection products which comprises a composition according to claim 1 or a pre-concentrate of emulsion therefor or a micellar solution wherein said preconcentrate or micellar solution comprises
a) a lipophilic phase, which comprises a composition according to claim 1 and
b) an aqueous phase.

17. Product according to claim 16, from the group of food-stuffs which contains co-enzyme Q10 or from the group of pharmaceuticals which contains insulin.
